(19) United States
(12) Patent Application Publication
(10) Pub. No.: US 2019/0192740 A1
(43) Pub. Date: Jun. 27, 2019

(54) ANTIBIOTIC COATING

(71) Applicant: Heraeus Medical GmbH, Wehrheim (DE)

(72) Inventors: Sebastian VOGT, Erfurt (DE); Hubert BÜCHNER, Nürnberg (DE); Klaus-Dieter KÜHN, Marburg-Elnhausen (DE)

(21) Appl. No.: 16/282,593

(22) Filed: Feb. 22, 2019

Related U.S. Application Data
(63) Continuation-in-part of application No. 13/110,096, filed on May 18, 2011.

(30) Foreign Application Priority Data
May 19, 2010 (DE) 10 2010 020 940.6

(51) Int. Cl. A61L 27/54; A61L 27/28

(52) U.S. Cl. CPC A61L 27/54; A61L 2300/406; A61L 27/28

(57) ABSTRACT

An implant is provided having an antibiotic coating that is inexpensive and easy to manufacture. The antibiotic coating adheres well and in stable manner to the surface of the implant, can be degraded by the body without forming toxic products, prevents the crystallization of the antibiotics in the coating, and ensures a high immediate locally-effective antibiotic concentration at the implant site. A coating solution useful for making the implant and a method for the manufacture of the implant are also described.

### CROSS-REFERENCE TO RELATED APPLICATIONS

This application is a continuation-in-part application claiming priority under 35 U.S.C. § 120 to U.S. patent application Ser. No. 13/110,096 filed May 18, 2011, which claims priority under 35 U.S.C. § 119 to the German Application No. 10 2010 020 940.6, filed May 19, 2010, which is hereby incorporated by reference herein in its entirety.

### BACKGROUND OF THE INVENTION

Embodiments of the present invention relate to an implant that comprises a coating, a coating solution that can be used to manufacture the implant, and a method to manufacture the implant.

Any implantation of articular endoprostheses, as well as osteosynthesis materials, is associated with a risk of microbial contamination. Successful colonization of the surface of the implant by microbial pathogens can lead to the manifestation of postoperative osteitis. Osteitis is a serious complication for the patient. Accordingly, there is a need to protect implants from bacterial colonization for the first hours to days after the implantation.

PMMA bone cement containing one or more antibiotics has been in clinical use with cemented articular endoprostheses for decades with much success. This development is based on the work of Buchholz and Engelbrecht (H. W. Buchholz and E. Engelbrecht, “Über die Depotwirkung einiger Antibiotika beim Vermischen mit dem Kunstharz Palacos,” [About the Repository Effect of Antibiotics upon mixing with the Synthetic Resin Palacos] Chirurg. [Surgery] 41: 511-515 (1970)). In this context, it is customary to use the broad-spectrum antibiotic, gentamicin, which protects the surface of the bone cement effectively against bacterial colonization and infections.

With regard to non-cemented articular endoprostheses and osteosynthesis materials, a number of approaches has been proposed in order to also attain local antibiotic protection of the implant surfaces. The approaches are based on the observation that antibiotics or conventional antibiotic salts adhere to the surface of implants either not at all or very poorly. To solve the problem, a number of published patent applications described the coating of implants with certain poorly water-soluble antibiotic salts as the components having the antibiotic activity. In this context, European patent application publications EP 0 623 349 A1, EP 1 470 829 A1, and EP 1 374 923 A2, and German published patent applications DE 101 42 465 A1, and DE 44 04 018 A1 shall be cited for exemplary purposes. Such poorly water-soluble antibiotic salts dissolve over time while releasing the antibiotics contained therein through exposure to the action of body fluids. However, it is disadvantageous in this context that the manufacture of the antibiotic salts requires much effort.

The need to use a poorly soluble form of an antibiotic or to convert the antibiotic to a poorly soluble form in order to attain a stable coating, results in delayed release of the antibiotic. However, it is often desirable to establish a high and effective antibiotic concentration at the site of implantation without delay.

Alternatively, it is feasible to use water-soluble antibiotic salts for coatings (V. Alt, A. Bitschnau, J. Osterling, A. Sewing, C. Meyer, R. Kraus, S. A. Meissner, S. Wenisch, E. Domann, R. Schnettler, “The effects of combined gentamicin-hydroxylapatite coating for cementless joint prostheses on the reduction of infection rates in a rabbit infection prophylaxis model,” Biomaterials 27 (26): 4627-34, (2006). One problem that has been evident in this context is that such antibiotics are difficult to fix in place on the implant surface. To solve the problem, Alt et al. incorporated gentamicin in a porous hydroxyl apatite coating that serves as a carrier material. However, a problem arising in this latter context is that the degradation of the coating in the body leads to the generation of toxic products.

Moreover, many antibiotics tend to crystallize. This is true in particular of, e.g., lincosamide, amikacin, and tobramycin. If such antibiotics are used to coat implants, there is a risk that the antibiotics might crystallize and damage surrounding tissues.

### BRIEF SUMMARY OF THE INVENTION

The invention therefore relates to the object to generate an implant having an antibiotic coating that is inexpensive and easy to manufacture, adheres well and in stable manner to the surface of the implant, can be degraded by the body without forming toxic products, prevents the crystallization of the antibiotics in the coating, and ensures high immediate locally-effective antibiotic concentration at the implant site. Moreover, the coating can adhere to surfaces that do not contain a porous hydroxyl apatite coating.

This object is met by an implant that comprises a coating that contains at least one antibiotic, water, and at least one humectant selected from the group consisting of water-soluble polyols, oligoalkylene glycols, and amino acids.

A coating solution containing at least one antibiotic, water, and at least one humectant selected from the group consisting of water-soluble polyols, oligoalkylene glycols, and amino acids can be used to meet the object, i.e., for the manufacture of the implant according to embodiments of the invention.

The implant according to embodiments of the invention can be manufactured by heating an implant to be coated to a temperature of at least about 90° C. and applying a coating solution according to an embodiment of the invention to the heated implant to be coated.

### DETAILED DESCRIPTION OF THE INVENTION

The invention is based on the surprising finding that antibiotics can form well-adhering, viscous, leather-like layers provided the layers contain small quantities of moisture. Humectants with a hygroscopic effect can be used to maintain a residual water content in the layers. This enables the antibiotic-containing layers to remain mechanically stable to a sufficient extent and not to disintegrate.

In the scope of the invention, the term “implants” shall be understood to mean materials and devices that can be introduced into the inside of the body, at least in part, in the course of a surgical intervention. The implants can contact the bone or other elements of the musculo-skeletal apparatus or be in contact with blood or connective tissue. The implants can, for example, be articular endoprostheses or osteosynthesis materials.

In the scope of the invention, the term “coating” shall be understood to mean a layer that covers at least one surface of the implant, at least in part. According to a preferred embodiment of the present invention, at least one surface of the implant is covered completely by a coating.

The coating contains at least one antibiotic. Preferably, the antibiotic is a water-soluble antibiotic.

In the scope of the invention, a “water-soluble antibiotic” shall be understood to mean an antibiotic whose solubility in water at a temperature of 25° C. is at least about 5 g/l, preferably at least about 7 g/l, and even more preferably at least about 10 g/l.

The antibiotic can be provided in any form in which the antibiotic has antibiotic efficacy or which enables the release of a compound having an antibiotic effect.

Therefore, according to embodiments of the invention, the term “antibiotic” also encompasses antibiotic salts or antibiotic esters as well as the corresponding hydrated forms of the antibiotic, antibiotic salts or antibiotic esters.

According to a preferred embodiment, the antibiotic can be selected from the group consisting of aminoglycoside antibiotics, polypeptide antibiotics, glycopeptides, β-lactams, polyketides, quinolones, and sulfonamides.

The aminoglycoside antibiotics include, but are not limited to, amikacin, apramycin, geneticin, gentamicin, kanamycin, netilmicin, neomycin, paromomycin, spectinomycin, streptomycin or tobramycin. According to a particularly preferred embodiment, the aminoglycoside antibiotic is gentamicin or a gentamicin derivative including, but not limited to, gentamicin salts or gentamicin esters. Gentamicin sulfate shall be mentioned as an exemplary gentamicin salt. Gentamicin sulfate is an inexpensive broad-spectrum antibiotic that has proven its utility in surgery and orthopaedics for decades. Pharmacopoeia-compliant gentamicin sulfate is a mixture of the gentamicin homologs, C1a, C1, C2a, C2b, and C2. Due to the presence of a mixture of multiple gentamicin homologs, this antibiotic does not crystallize. Compared to other antibiotics, gentamicin has a special feature in that it can tolerate elevated temperatures for brief periods of time without loss of its antimicrobial efficacy.

Polypeptide antibiotics that can be used in embodiments of the present invention include, but are not limited to, polymyxins, bacitracin, and tyrothricin.

β-lactams that can be used in embodiments of the present invention include, but are not limited to, penicillins, cephalosporins, monobactams, and carbapenems.

Examples of polyketides that can be used in embodiments of the present invention include, but are not limited to, tetracyclins or macrolide antibiotics, such as, for example, erythromycin.

Moreover, the coating contains a humectant. The humectant meets the objective of setting a desired residual moisture content for the coating. According to embodiments of the invention, a compound selected from the group consisting of water-soluble polyols, oligoalkylene glycols, and amino acids can be used as the humectant.

According to embodiments of the invention, “polyols” are understood to be low-molecular compounds that comprise at least two hydroxyl groups.

According to embodiments of the invention, the polyols contain at least two carbon atoms. The polyols according to embodiments of the invention preferably comprise 2-50, more preferably 2-25, even more preferably 2-15, particularly preferably 2-10, more particularly preferably 2-8, and in particular 2-6 carbon atoms.

According to embodiments of the invention, the polyols can be linear or cyclic in structure, preferably they are linear in structure.

Moreover, the polyols can be branched or non-branched, preferably they are non-branched.

Moreover, the polyols can be chiral or achiral.

The polyols according to embodiments of the invention can contain at least two, at least three, at least four or at least five hydroxyl groups. Preferably, the polyols comprise 2-15, more preferably 2-10, even more preferably 2-7, particularly preferably 2-5, more particularly preferably 2-4, and in particular 2 or 3 hydroxyl groups.

The hydroxyl groups of the polyols can be arranged on one or more carbon atom(s). According to a preferred embodiment, each carbon atom of a polyol according to the invention contains at most one hydroxyl group. The hydroxyl group-bearing carbon atoms can be neighboring with respect to each other or separated from each other by at least one, at least two, at least three or at least four carbon atoms. According to a particularly preferred embodiment, each carbon atom of the polyol according to the invention bears one hydroxyl group.

The hydroxyl groups of the polyols according to embodiments of the invention are preferably independent functional groups. Accordingly, the carbon atoms bearing the at least one hydroxyl group comprise no other bonds to hetero atoms, such as, for example, oxygen atoms, nitrogen atoms or sulfur atoms, aside from the bonds to the oxygen atoms of the hydroxyl groups. Preferably, said hydroxyl groups are therefore not part of other functional groups such as, for example, carbonic acid groups. However, according to embodiments of the invention, the polyols can comprise, on carbon atoms bearing at least one hydroxyl group, a bond to a neighboring carbon atom that is a component of a functional group, for example a carbonic acid group or an ester group, or comprises a bond to a functional group or a hetero atom.

The polyols according to embodiments of the invention can be low-molecular. In the scope of the invention, the polyols are low-molecular if their molecular mass is less than about 5,000 g/mol, preferably less than about 3,000 g/mol, even more preferably less than about 2,000 g/mol, particularly preferably less than about 1,000 g/mol, and most particularly preferably less than about 500 g/mol.

According to embodiments of the invention, water-soluble polyols are understood to be polyols whose solubility in water at a temperature of 25° C. is at least about 5 g/l, preferably at least about 7 g/l, and even more preferably at least about 10 g/l.

According to a preferred embodiment, the polyols according to the invention are alditols.

According to embodiments of the invention, alditols are understood to be non-cyclic polyols represented by formula (I)

HOCH₂[CH(OH)]ₙCH₂OH (I)

wherein n is an integer. Preferably, n is an integer from 1-10, more preferably from 1-8, even more preferably from 1-7, and particularly preferably from 1-6.

According to a particularly preferred embodiment, the polyols according to the invention are selected from the group consisting of glycerol, sorbitol, mannitol, glucitol, isomalt, lactite, xylitol, threitol, erythritol, arabitol, 1,2-ethanediol, 1,2-propanediol, and dianhydroglycitol.

According to embodiments of the invention, oligoalkylene glycols are understood to mean oligomeric compounds represented by formula (II)

—R1—O—X1—O—R2— (II)

wherein R1 and R2, independent of each other, are a substituted or non-substituted, linear or cyclic, branched or non-branched alkyl residue that preferably comprises 1-10 carbon atoms, more preferably 1-5 carbon atoms, and most preferably 1-3 carbon atoms, and X1 is a structural unit that comprises at least one ether group, but preferably at most 100 ether groups, more preferably at most 25 ether groups, even more preferably at most 20 ether groups, and particularly preferably at most 10 ether groups that are formed by alkyl residues of equal or different structure having preferably 1-10 carbon atoms each, more preferably 1-5 carbon atoms each, and most preferably 1-3 carbon atoms each, which each are connected to each other through an oxygen atom each.

The oligoalkylene glycols according to embodiments of the invention have a molecular mass of less than about 5,000 g/mol, preferably less than about 3,000 g/mol, even more preferably less than about 2,000 g/mol, particularly preferably less than about 1,000 g/mol, and most preferably less than about 500 g/mol.

According to a preferred embodiment, the oligoalkylene glycol is an oligoethylene glycol or an oligopropylene glycol.

According to embodiments of the invention, oligoethylene glycols are compounds that are represented by formula (III)

H—[OCH₂CH₂]ₘ—OH (III)

wherein m is an integer from 1-100, preferably from 1-25, more preferably from 1-20, even more preferably from 1-10, and particularly preferably from 1-5.

Oligopropylene glycols are compounds that are represented by formula (IV)

H—[OCH₂(CH₃)CH₂]ₗ—OH (IV)

wherein 1 is an integer from 1-100, preferably from 1-25, more preferably from 1-20, even more preferably from 1-10, and particularly preferably from 1-5.

According to a particularly preferred embodiment, the oligoalkylene glycol is selected from the group consisting of ethylene glycol, diethylene glycol, triethylene glycol, tetraethylene glycol, propylene glycol, dipropylene glycol, tripropylene glycol, and tetrapropylene glycol.

Moreover, the coating of the implant can comprise an amino acid.

The amino acid can be an essential or a non-essential amino acid.

The amino acid can be an aliphatic, aromatic or heterocyclic amino acid, whereby aliphatic amino acids are preferred.

Preferably, the amino acid has a molar mass in the range of about 75-200 g/mol.

According to a particularly preferred embodiment, the amino acid is glycine.

While not wishing to be bound by theories, it is believed that the effect of the humectants according to embodiments of the invention is that a residual moisture content is retained in the coating of the implant and the antibiotic thus adheres to substrates, preferably metal surfaces, as a viscous, amorphous layer. Moreover, some residual moisture being retained can effectively prevent the antibiotics from crystallizing.

The water fraction of the coating, relative to the mass of the implant coating, preferably is in the range of about 5-30 weight percent and even more preferably in the range of about 7-28 weight percent.

Upon implantation of the implant according to embodiments of the invention, the coating dissolves within a few minutes to hours under the influence of body fluids, such as blood and wound exudations. This ensures that the immediate antibiotic concentration at the site of implantation is high and efficacious. The antibiotic contained in the coating is released right at the boundary between implant and bone tissue or soft tissue and thus effectively protects the implant surface from bacterial colonization.

According to embodiments of the invention, the coating of the implant can contain additional components. For example, the coating can comprise at least one more antiseptic or hemostyptic agent.

The term, “antiseptic agent”, is meant to encompass all antiseptic substances that are common in medicine. In this context, octenidine dihydrochloride, polyhexanide and quaternary ammonium compounds, such as, for example, benzalkonium chloride, are particularly preferred.

The term, “hemostyptic agent”, shall be understood to mean substances that activate the coagulation of blood. Calcium salts, in particular, are preferred as hemostyptic agents. According to a preferred embodiment, the calcium salt is selected from the group consisting of calcium chloride, calcium acetate, and calcium lactate.

According to embodiments of the invention, a cover layer can be arranged on the coating of the implant. The cover layer preferably contains biocompatible film-forming agents. Film-forming agents are compounds that are capable of forming a film layer on a surface. According to a preferred embodiment, film-forming antibiotic salts, antibiotic esters, antiseptic salts and/or antiseptic esters can be used as film-forming agents. However, other film-forming agents can be used as well, in particular polymeric film-forming agents, such as, for example, methyl cellulose, polyvidone acetate, hydroxypropylmethylcellulose phthalate, carboxymethyl cellulose, polyvinylacetate phthalate, shellack or methacrylic acid esters.

The coating of the implant according to an embodiment of the invention preferably contains about 0.01-80 weight percent of the at least one antibiotic, about 0.01-80 weight percent of the at least one humectant, about 5-30 weight percent of water, and about 0-50 weight percent of other components, with respect to the weight of the coating.

According to a preferred embodiment, the weight ratio of the at least one antibiotic and the at least one humectant is in the range of about 1,000:1 to 1:1.

The manufacture of the implant according to embodiments of the invention can include the use of a coating solution. The coating solution comprises at least one antibiotic, water, and at least one humectant selected from the group consisting of water-soluble polyols, oligoalkylene glycols, and amino acids.

With respect to the at least one antibiotic of the coating solution and the at least one humectant, reference shall be made to the explanations provided above.

According to a preferred embodiment, the coating solution contains about 0.01-40 weight percent of the at least one antibiotic, about 0.01-40 weight percent of the at least one humectant, about 10-99.5 weight percent of water, and about 0-50 weight percent of other components.

The implant according to embodiments of the invention can be manufactured by a variety of routes in view of the present disclosure.

According to an embodiment of the invention, the coated implant can be manufactured by first providing the implant to be coated and then heating it to a temperature of at least about 90° C., preferably at least about 100° C.

Subsequently, the coating solution according to an embodiment of the invention can be applied to the heated implant to be coated.

In this context, it has proven advantageous to heat the coating solution to a temperature of more than about 40° C., preferably of more than about 50° C., right before application onto the implant. By this means, the water contained in the coating solution evaporates rapidly upon application to the heated substrate.

The coating solution can be applied to the implant, for example, through spraying. When the spray mist hits the heated substrate, virtually all of the water evaporates. In the process, the at least one antibiotic and the at least one humectant remain as coating on the implant, whereby the presence of the at least one humectant causes part of the water from the coating solution to be retained in the coating. Thus, a horn-like layer is formed on the substrate.

If applicable, the coating can be subjected to a process of subsequent drying afterwards. Said subsequent drying can be effected in a stream of warm air or in a vacuum. Moreover, it is feasible just as well to dry the coating through the action of microwave radiation. During the coating of metallic implants, it is also feasible to perform inductive heating of the implants through the action of alternating magnetic fields in order to dry the coating rapidly.

If the coated implant is to be provided with a cover layer, a powdered film-forming agent, for example, powdered antibiotics and/or antiseptics, can be applied to the coating, for example during the drying of the coating, while it is still sticky. The film-forming agent can be applied by spraying or immersing in a bath containing the powdered agent.

Another option for providing the coated implant with a cover layer is to apply a solution of a film-forming agent, for example, an alcoholic solution of an antibiotic, onto the coated implant. For this purpose, the coated implant is preferably first heated to a temperature of at least about 90° C., more preferably at least about 100° C., and the solution of the film-forming agent is subsequently applied onto the coated implant while the solvent evaporates.

### EXAMPLES

The invention is illustrated in more detail through the examples presented in the following, though without limiting the scope of the invention.

### Example 1

A total of 20 g gentamicin sulfate (made by Fujian Fukang Ltd., China) and 0.1 g glycerol were dissolved in 80 g of water. A clear, weakly yellow solution was thus produced. Using a pneumatic spray gun, said solution was then sprayed onto a titanium disc (1.5 cm diameter) that had been heated to 120° C. earlier. Part of the water evaporated and an even, horn-like layer was formed on the titanium disc. The coated titanium disc was dried at 100° C. in a drying cabinet until the mass remained constant, and the mass of the coating was determined through gravimetry. The mass of the coating was 1 mg.

### Example 2

A total of 20 g gentamicin sulfate (made by Fujian Fukang Ltd., China), 0.1 g triethylene glycol (Fluka), and 0.05 g calcium chloride (Fluka) were dissolved in 80 g of water. A clear, weakly yellow solution was thus produced. Using a pneumatic spray gun, said solution was then sprayed onto a titanium disc (1.5 cm diameter) that had been heated to 120° C. earlier. Part of the water evaporated and an even, horn-like layer was formed on the titanium disc. The coated titanium disc was dried at 100° C. in a drying cabinet until the mass remained constant, and the mass of the coating was determined through gravimetry. The mass of the coating was 0.9 mg.

### Example 3

A total of 20 g gentamicin sulfate (made by Fujian Fukang Ltd., China), 0.05 g glycerol (Fluka), and 0.05 g benzalkonium chloride were dissolved in 80 g of water. A clear, weakly yellow solution was thus produced. Using a pneumatic spray gun, said solution was then sprayed onto a titanium disc (1.5 cm diameter) that had been heated to 120° C. earlier. Part of the water evaporated and an even, horn-like layer was formed on the titanium disc. The coated titanium disc was dried at 100° C. in a drying cabinet until the mass remained constant, and the mass of the coating was determined through gravimetry. The mass of the coating was 0.9 mg.

### Example 4

A total of 20 g gentamicin sulfate (made by Fujian Fukang Ltd., China), 0.05 g glycerol (Fluka), 0.05 g benzalkonium chloride, and 0.05 g calcium chloride were dissolved in 80 g of water. Using a pneumatic spray gun, said solution was then sprayed onto a titanium disc (1.5 cm diameter) that had been heated to 120° C. earlier. Part of the water evaporated and an even, horn-like layer was formed on the titanium disc. The coated titanium disc was dried at 100° C. in a drying cabinet until the mass remained constant, and the mass of the coating was determined through gravimetry. The mass of the coating was 1.0 mg.

### Example 5

A total of 20 g gentamicin sulfate (made by Fujian Fukang Ltd. China) and 0.05 g glycerol (Fluka) were dissolved in 80 g of water. A clear, weakly yellow solution was thus produced. Using a pneumatic spray gun, said solution was then sprayed onto a titanium disc (1.5 cm diameter) that had been heated to 120° C. earlier. Part of the water evaporated and an even, horn-like layer was formed on the titanium disc. The coated titanium disc was dried at 100° C. in a drying cabinet until the mass remained constant, and the mass of the coating was determined through gravimetry. The mass of the coating was 0.9 mg. Subsequently, the dried, coated titanium disc was heated to 100° C. and sprayed with a 4% methanolic gentamicin palmitate solution. The methanol evaporated in the process and a cover layer made of gentamicin palmitate was formed. After drying until the mass remained constant, the titanium disc thus coated was weighed again. The mass of the cover layer was 1.3 mg.

### Example 6

A total of 20 g gentamicin sulfate (made by Fujian Fukang Ltd. China) and 0.05 g glycerol (Fluka) were dissolved in 80 g of water. A clear, weakly yellow solution was thus produced. Using a pneumatic spray gun, the solution was then sprayed onto a titanium disc (1.5 cm diameter) that had been heated to 120° C. earlier. Part of the water evaporated and a coating layer had been formed on the titanium disc. To vary the water content in this coating layer, the disc was placed in a drying cabinet at 100 ° C. for varying periods of time after the initial coating step. The longer the drying step, the less water remained in the coating layer. The water content of the coating layer was measured using Karl-Fischer-titration. The consistency of the coating layer was inspected with the naked eye, after scratching the coating with a spatula. The glycerol content was measured by drying the coating layer in a drying cabinet at 150° C. under reduced pressure to constant weight. The drying loss corresponds to the glycerol content, considering the previously determined water content.

As shown by the results summarized in the following table, when the water content in the coating was too low, e.g., 2% wt. % or 4% wt. %, the coating was too brittle to properly stick to the implant. On the other hand, when the coating contained too much water, e.g., 30% wt. % or 35% wt. %, the coating became too soft and sticky, and also did not form a proper coating.

| Water content wt. % | Glycerol wt. % | Consistency |
|---|---|---|
| 2 | 3 | Brittle |
| 4 | 3 | Brittle |
| 7 | 3 | leather-like |
| 12 | 3 | leather-like |
| 16 | 3 | leather-like |
| 20 | 3 | leather-like |
| 24 | 3 | leather-like |
| 28 | 3 | leather-like |
| 30 | 3 | Soft |
| 35 | 3 | soft, viscous |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of preparing a coated implant having a leather-like or horn-like coating, the method comprising:
(i) heating an implant to a temperature of at least about 90° C. to obtain a heated implant;
(ii) heating a coating solution to a temperature of more than 40° C. to obtain a heated coating solution, and
(iii) applying the heated coating solution to the heated implant to form the leather-like or horn-like coating on the implant,
wherein the leather-like or horn-like coating comprises:
(a) at least one antibiotic,
(b) 7 to 28% by weight of water, and
(c) at least one humectant selected from the group consisting of (c1) water-soluble polyols having a molecular mass of less than 5,000 g/mol, selected from the group consisting of glycerol, sorbitol, mannitol, glucitol, isomalt, lactite, xylitol, threitol, erythrol, arabitol, 1,2-ethanediol, 1,2-propanediol, and dianhydroglycitol, (c2) oligoalkylene glycols, selected from the group consisting of ethylene glycol, diethylene glycol, triethylene glycol, and tetraethylene glycol, and (c3) amino acids,
wherein the weight ratio of the at least one antibiotic to the at least one humectant is 1,000:1 to 1:1.

2. The method according to claim 1, wherein the antibiotic is a water-soluble antibiotic.

3. The method according to claim 2, wherein the water-soluble antibiotic is a gentamicin salt.

4. The method according to claim 3, wherein the gentamicin salt is gentamicin sulfate.

5. The method according to claim 1, wherein the humectant is a water-soluble polyol selected from the group consisting of glycerol, sorbitol, mannitol, glucitol, and dianhydroglycitol.

6. The method according to claim 1, wherein the humectant is an oligoalkylene glycol selected from the group consisting of ethylene glycol, diethylene glycol, triethylene glycol, and tetraethylene glycol.

7. The method according to claim 1, wherein the humectant is glycine.

8. The method according to claim 1, wherein the leather-like or horn-like coating further comprises at least one additional substance selected from the group consisting of antiseptics, antibiotics, antiphlogistic agents, and hemostyptic agents.

9. A method of preparing a coated implant having a leather-like or horn-like coating, the method comprising:
(i) heating an implant to a temperature of at least about 90° C. to obtain a heated implant;
(ii) applying a coating solution to the heated implant to form a coating on the implant, and
(iii) partially drying the coating to form the leather-like or horn-like coating on the implant,
wherein the leather-like or horn-like coating comprises:
(a) at least one antibiotic,
(b) 7 to 28% by weight of water, and
(c) at least one humectant selected from the group consisting of (c1) water-soluble polyols having a molecular mass of less than 5,000 g/mol, selected from the group consisting of glycerol, sorbitol, mannitol, glucitol, isomalt, lactite, xylitol, threitol, erythrol, arabitol, 1,2-ethanediol, 1,2-propanediol, and dianhydroglycitol, (c2) oligoalkylene glycols, selected from the group consisting of ethylene glycol, diethylene glycol, triethylene glycol, and tetraethylene glycol, and (c3) amino acids,
wherein the weight ratio of the at least one antibiotic to the at least one humectant is 1,000:1 to 1:1.

10. The method according to claim 9, wherein the antibiotic is a water-soluble antibiotic.

11. The method according to claim 10, wherein the water-soluble antibiotic is a gentamicin salt.

12. The method according to claim 11, wherein the gentamicin salt is gentamicin sulfate.

13. The method according to claim 9, wherein the humectant is a water-soluble polyol selected from the group consisting of glycerol, sorbitol, mannitol, glucitol, and dianhydroglycitol.

14. The method according to claim 9, wherein the humectant is an oligoalkylene glycol selected from the group consisting of ethylene glycol, diethylene glycol, triethylene glycol, and tetraethylene glycol.

15. The method according to claim 9, wherein the humectant is glycine.

16. The method according to claim 9, wherein the leather-like or horn-like coating further comprises at least one additional substance selected from the group consisting of antiseptics, antibiotics, antiphlogistic agents, and hemostyptic agents.

17. The coated implant having the leather-like or horn-like coating prepared by the method of claim 1.

18. The coated implant having the leather-like or horn-like coating prepared by the method of claim 9.

19. A coated implant having a leather-like or horn-like coating, wherein the coating comprises:
(a) at least one antibiotic,
(b) 7 to 28% by weight of water, and
(c) at least one humectant selected from the group consisting of (c1) water-soluble polyols having a molecular mass of less than 5,000 g/mol, selected from the group consisting of glycerol, sorbitol, mannitol, glucitol, isomalt, lactite, xylitol, threitol, erythrol, arabitol, 1,2-ethanediol, 1,2-propanediol, and dianhydroglycitol, (c2) oligoalkylene glycols, selected from the group consisting of ethylene glycol, diethylene glycol, triethylene glycol, and tetraethylene glycol, and (c3) amino acids,
wherein the weight ratio of the at least one antibiotic to the at least one humectant is 1,000:1 to 1:1.

20. The coated implant of claim 19, wherein the at least one antibiotic comprises gentamicin sulfate, the at least one humectant comprises glycerol or triethylene glycol, and the leather-like or horn-like coating optionally further comprises benzalkonium chloride.
